(19) United States
(12) Patent Application Publication
(10) Pub. No.: US 2022/0170924 A1
(43) Pub. Date: Jun. 2, 2022

(54) NANOSCALE MOLECULAR AND IMUNO-ASSAY SENSING USING SYMMETRY-BREAKINGINDUCED PLASMONIC EXCEPTIONAL POINTS

(71) Applicant: THE REGENTS OF THE UNIVERSITY OF CALIFORNIA, Oakland, CA (US)

(72) Inventors: Abdoulaye NDAO, La Jolla, CA (US); Jun-Hee PARK, La Jolla, CA (US); Boubacar KANTE, La Jolla, CA (US)

(21) Appl. No.: 17/441,821

(22) PCT Filed: Mar. 25, 2020
(86) PCT No.: PCT/US20/24658

§ 371 Date: Sep. 22, 2021

Related U.S. Application Data
(60) Provisional application No. 62/823,158, filed on Mar. 25, 2019.

(51) Int. Cl. G01N 33/543; G01J 9/02

(52) U.S. Cl. CPC G01N 33/54373; B82Y 35/00; G01J 9/02

(57) ABSTRACT

A method for detecting an analyte includes providing a sensor that includes a plurality of coupled polaritonic structures having polaritonic resonances. A surface of at least one of the polaritonic structure in the sensor is functionalized by providing a receptor for binding the analyte to the surface. The sensor is operated at an exceptional point (EP). The presence of the analyte on the surface is identified when a degeneracy of resonant frequencies and linewidths is lifted and a splitting of the resonant frequencies and linewidths occurs.

### CROSS-REFERENCE TO RELATED APPLICATION

This application claims the benefit of U.S. Provisional Application Ser. No. 62/823,158, filed Mar. 25, 2019, the contents of which are incorporated herein by reference. This application is also related to U.S. application Ser. No. 16/331,177, filed Mar. 7, 2019 (Our Ref : 22000/31).

### BACKGROUND

Sensing is fundamental to our observation of the universe via physical quantities such as mass, time, or distances. Molecular nanosensing, i.e., the ability to detect extremely small quantities, enables the detection of threats at early stage and will revolutionize security and medicine. Sensing technologies in classical and quantum regimes are usually based on non-destructive probing utilizing enhanced wave-matter interaction at resonance. The interaction of waves with a sensor thus requires the latter to be an open system, i.e., a non-Hermitian system described by both radiative and absorptive processes.

Molecular level sensors are central to the identification of numerous nanoscale substances such as harmful biological pathogens, air-borne toxins, pesticides, water contaminants, tumor markers, brain diseases, or for blood-glucose concentration. As such, the need for low-cost nanoscale sensing is paramount especially since billions of dollars are spent every year on medical diagnostics alone (ex. blood tests). Sensing microvolt levels has always proven to be a great challenge as it requires a sensor with a very high degree of sensitivity yet with nanoscale dimensions. Currently, there are various configurations for molecular sensors ranging from semiconductor nanowire arrays to optical micro toroid resonators. The quantitative variation is subsequently used to identify the target molecule and its concentration. In both cases, an external perturbation of δ caused by the target molecule will lead to a change proportional to δ, thus resulting in a limited sensitivity. For an increased sensitivity, these sensors can be vastly improved by employing resonant coupled plasmonic particles operating at so-called exceptional points or all dielectric bound states in the continuum (high Q).

Recently, non-Hermitian singularities known as exceptional points (EPs) have been observed in systems including electromagnetism, atom-cavity, and acoustics. EPs are singularities where at least two eigenmodes of an open system coalesce to become degenerate both in their resonance frequencies and decay rates, i.e. linewidths. At such singularities, the topology of the system is drastically modified and it appears skewed with reduced dimensionality but enhanced sensitivity. To date, the observation of EPs has been restricted to wavelength scaled systems based on dielectric waveguides and resonators subject to diffraction limit. While PT symmetry prescribes a systematic recipe to implement EPs in those systems, its implementation at subwavelength scales, in plasmonics, constitutes a formidable challenge requiring the controlled spatial distribution of loss and gain with extremely high precision. The observation of such non-Hermitian singularities at subwavelength scale has thus remained elusive.

This Background is provided to introduce a brief context for the Summary and Detailed Description that follow. This Background is not intended to be an aid in determining the scope of the claimed subject matter nor be viewed as limiting the claimed subject matter to implementations that solve any or all of the disadvantages or problems presented above.

### SUMMARY

Systems and methods according to present principles meet the needs of the above in several ways. In particular, what are disclosed are materials, designs, devices, systems and applications that pertain to ultrasensitive, molecular-level sensors that use exceptional points (EPs) singularities exhibited by non-Hermitian polaritonic, plasmonic and dielectric systems. Systems and methods according to present principles thus open a new class of compact nanoscale sensors and imagers with a wide range of applications.

In more detail, EPs, where at least two complex eigenmodes coalesce and manifest via simultaneous degeneracy of resonant frequencies and linewidths, are highly sensitive to external perturbations as even a tiny variation will lift the degeneracy and cause splitting of both resonant frequencies and linewidths.

Systems and methods according to present principles include the use of EPs in plasmonics, based on the hybridization of detuned resonators in a multilayered plasmonic crystal. Plasmons shrink the wavelength of light to make it compatible with biological relevant substances. BICs can confine light at microscale thus overcoming size limitations to high Quality factor of guided resonance modes in photonic crystals and enabling high field intensity in small volumes that can be exploited to realize high performance sensors.

Plasmonic EPs can be systematically implemented by controlling the interplay between near-field and far-field couplings in hybridized systems governed by Coulomb interactions and interferences respectively. The plasmonic metamaterial EP crystal described herein, made of passive coupled arrays of plasmonic resonators with detuned resonances, exhibit the topology of exceptional points around the non-Hermitian singularity and enhanced immuno-assay nanosensing was observed.

This Summary is provided to introduce a selection of concepts in a simplified form. The concepts are further described in the Detailed Description section. Elements or steps other than those described in this Summary are possible, and no element or step is necessarily required. This Summary is not intended to identify key features or essential features of the claimed subject matter, nor is it intended for use as an aid in determining the scope of the claimed subject matter. The claimed subject matter is not limited to implementations that solve any or all disadvantages noted in any part of this disclosure.

### BRIEF DESCRIPTION OF THE DRAWINGS

FIG. 1A is a schematic diagram of the unit cell of a structure with three paired gold nanobars supporting hybridized modes.

FIG. 1B illustrates an energy level diagram describing the plasmon hybridization in the gold-bar system of FIG. 1A.

FIGS. 2A-2H show a sequence of steps in one exemplary process for fabricating the structure shown in FIG. 1A.

FIG. 3 is a schematic diagram of the unit cell of a structure similar to that shown in FIG. 1A except the unit cell has only two paired gold nanobars instead of the three paired gold bars as in FIG. 1A

FIG. 4a is a schematic diagram of one example of a sensor that employs a bilayer plasmonic crystal similar to the structure shown in FIG. 3; FIG. 4b (left) presents a top-view scanning electron micrograph (SEM) of the fabricated bilayer structure, with a lateral shift between bars dx=100 nm and FIG. 4b (right) shows zoom-in top (XY-plane) and side (XZ-plane) views of the structure.

FIGS. 5a-5f illustrates a plasmonic exceptional point and symmetry-dependent hybridization scheme of resonances and loss rates for various configurations; FIG. 5g presents numerical simulations of nanosensor structures supporting a plasmonic EP and DP (both in configuration 3); FIG. 5h are log plots that confirm that the nanosensor operating around an EP exhibits resonance splitting (Δω) proportional to the square-root of the perturbation Δω_{EP}˜\sqrt{δ} whereas the DP nanosensor exhibits resonance splitting that depends linearly on the perturbation Δω_{DP}˜δ.

FIGS. 6a-6d presents experimental (circles) and simulated (dashed curves) resonance frequencies (ω) and loss rates (γ); FIGS. 6e and 6f show the absolute value of the out-of-plane component of the magnetic field of the modes (|Hy|) for P_{y}=400 nm and for P_{y}=430 nm, respectively, at indicated points on the resonance frequency dispersion plots.

FIG. 7 shows a sequence of steps in a process to functionalize with anti-Mouse IgG the top gold bars in DP and EP nanosensors.

FIG. 8a are histograms presenting the measured resonance splitting for different concentrations of anti-Mouse IgG for the Diabolic Point (DP) and Exceptional Point (EP) sensors; FIG. 8b shows the resonance splitting as a function of IgG concentration that can be regarded as an index perturbation.

Like reference numerals refer to like elements throughout. Elements are not to scale unless otherwise noted.

### DETAILED DESCRIPTION

Singularities, such as exceptional points, are fundamental in physics due to their uncanny ability to induce a large response from a small excitation. Singularities occur when a quantity is undefined or infinite, such as the density at the center of black hole, for example. Exceptional points occur when two waves become degenerate, meaning that both their resonant frequencies and spatial structure merge as one.

Exceptional points have been highly sought after for sensors and enhanced light-matter interactions. The possibility to demonstrate exceptional points in systems that are simultaneously sub-wavelength and compatible with small biological molecules for sensing has remained elusive until the development of present systems and methods.

Nanosensors operate based on a phenomenon called frequency splitting, meaning that the presence of a substance perturbs the degeneracy between two resonant frequencies and causes a detectable split. In an exceptional-point-based nanosensor, resonant frequencies would split much faster than they do in traditional nanosensors, giving rise to enhanced detection capabilities.

By combining exceptional points and plasmonics, present systems and methods provide a design for a nanosensor that is both compact and ultra-sensitive. Such a nanosensor is not just a gradual improvement of existing devices, but a conceptual breakthrough, and provides a general recipe to obtain exceptional points on demand.” The method involves controlling the interaction between symmetry-compatible modes of the plasmonic system.

Exceptional Points (EPs) are highly sensitive to external perturbations as even a tiny variation will lift the degeneracy and cause splitting of both resonant frequencies and linewidths. This is different from a regular shift in the resonant frequency of a resonator. Indeed, if a system operating at an EP is subjected to a perturbation of strength δ then its response, instead of being also of strength δ as in most sensors, is proportional to √δ. For a really small perturbation, where δ<<1, this characteristic square root dependence can drastically enhance the frequency splitting.

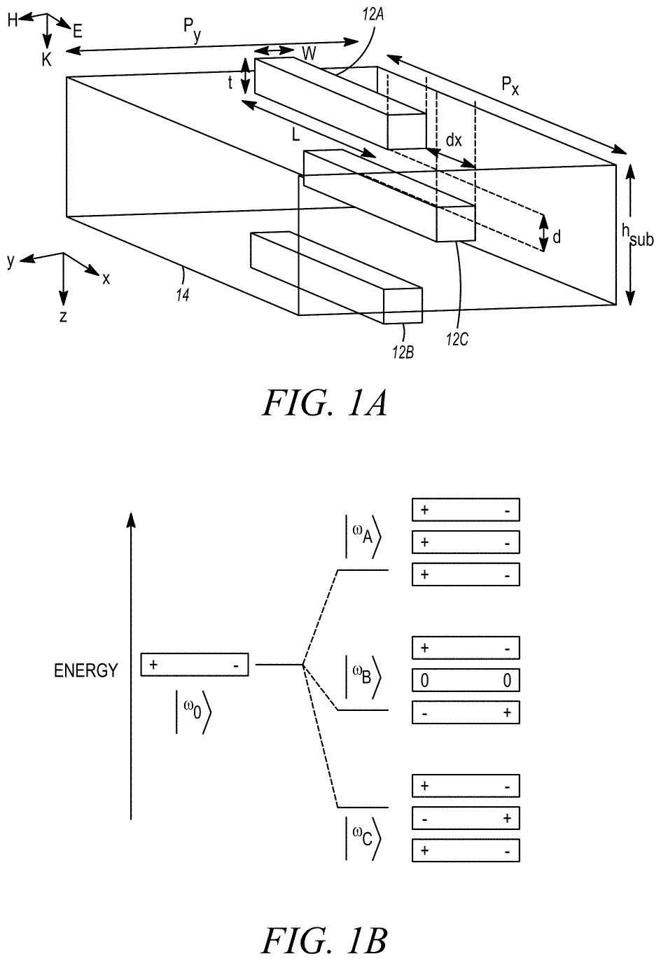

FIG. 1A is a schematic diagram of the unit cell of a structure with three paired gold nanobars 12a, 12b, and 12c, supporting hybridized modes, with the middle one 12c separated by a variable distance (dx, dy, dz) with respect to the other two 12a and 12b. A dielectric spacer 14 is shown, e.g., SiO₂. Exemplary dimensions of each nanobar may be, e.g., L (450 nm), W (50 nm), and T (40 nm), but it will be understood that these can vary.

The periodicity in x and y-directions are given by Px (800 nm) and Py (400 nm). The gold bars are described using a Drude model with a plasma frequency (ωp=1.367×1016 rad/sec) and collision frequency (ωc=6.478×1013 rad/sec).

FIG. 1B illustrates an energy level diagram describing the plasmon hybridization in the gold-bar system with three modes: ω_{A}, ω_{B}, ω_{C} where ω_{A}>ω_{B}>ω_{C} for dx=0. ω₀ corresponds to the resonance of an individual bar. As illustrated in FIG. 1A, systems and methods according to present principles employ coalescence of hybridized modes of the plasmonic system. The hybridized modes and their symmetries are portrayed on the energy scale in FIG. 1B. It is noted that this EP singularity is not limited by the number of plasmonic resonators, and EP's can be realized with higher numbers of resonators.

The instantaneous charge profiles of the first three modes are depicted in FIG. 1B. Intrinsically, the system has reflection symmetry with respect to the xy-plane that bisects the central nanobar and its modes are thus either even or odd. In this case, modes A and C have an even symmetry whereas Mode B has an odd symmetry. Mode A, with eigenfrequency ωA, has charges in all the bars oscillating in-phase and mode C, with eigenfrequency ωC, has charges in all bars oscillating out-of-phase. Mode B, ωB, has no charges in the central bar as seen in FIG. 1B. Therefore, mode A resides at a higher energy (higher frequency) due to all repelling Coulomb interactions and mode C resides at a lower energy (lower frequency) as a result of Coulomb interactions. Lastly, mode B resides between mode A and mode C on the energy scale.

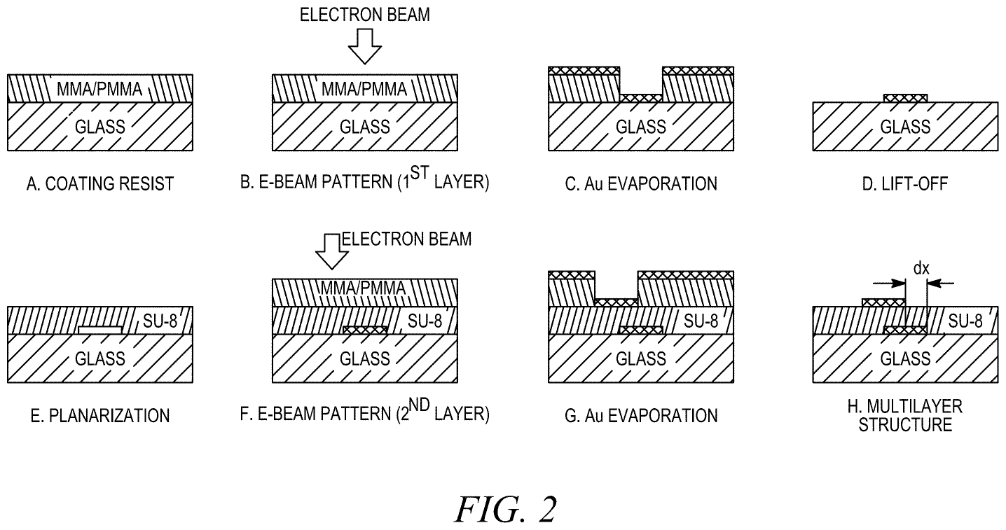

One exemplary fabrication process for this multi-layer structure is detailed in FIG. 2. Referring to FIGS. 2A-2D, starting with a clean glass substrate, MMA and PMMA are used as the bi-layer e-beam resist for the lithography. Au/Cr (37 nm/3 nm) metals are evaporated after resist development followed by a lift-off process completing the first layer of the metasurface. FIG. 2E shows how SU-8 is spun on to the first layer acting as a dielectric spacer between layers. However, the surface of the SU-8 layer is uneven due to the existence of the first layer and is planarized by thermally cycling the sample repeatedly followed by SU-8 crosslinking via UV light exposure plus hard baking. FIG. 2F-2H shows how E-beam lithography, metallization and lift-off steps are repeated for the second layer to realize the completed multi-layer structure.

In more detail, the multilayer metamaterials are fabricated on a glass substrate using high-resolution electron-beam lithography (EBL) (Vistec EBPG5200 writer). First, the glass substrate is cleaned with acetone and isopropyl alcohol (IPA) while sonicating. To minimize sidewall roughness during the lift-off process, high-resolution positive-tone bilayer resists, methyl methacrylate (MMA-EL 8) and polymethyl methacrylate (PMMA-A2) are used for the e-beam resist. MMA resist is spun on first at a thickness of 150 nm and 50 nm of PMMA is spun subsequently (FIGS. 2A and 2B). After the writing step and development by MIBK solvent, a 3 nm layer of chromium (adhesion layer) is deposited followed by 37 nm of gold (Au) using an electron beam evaporation system. The e-beam resist is lifted off using a photoresist remover, completing the first layer (FIGS. 2C and 2D).

After the lift-off process, a 100 nm thick SU-8 photoresist is spin-coated onto the sample. Due to the existence of the first layer of metallic structures, the surface of the SU-8 layer is uneven and needs to be planarized for subsequent fabrication steps. This is done by thermally cycling the sample repeatedly followed by SU-8 crosslinking via UV light exposure and a final hard bake step. To confirm the planarization, the roughness of SU-8 layer surface was determined using atomic force microscopy (AFM) and the surface roughness (RMS) was found to be below 5 nm. Thus, the first layer of gold bars on the glass substrate are embedded in SU-8 which also serves as a dielectric spacer (FIG. 2E). EBL, metal deposition, and lift-off steps for the second layer are carried out in a similar manner as the first layer with the requirement of gold alignment marks to ensure the precise stacking of layers (FIGS. 2F-2H). The completed multilayer structure can be seen in FIG. 2H. More layers can be added by repeating the process.

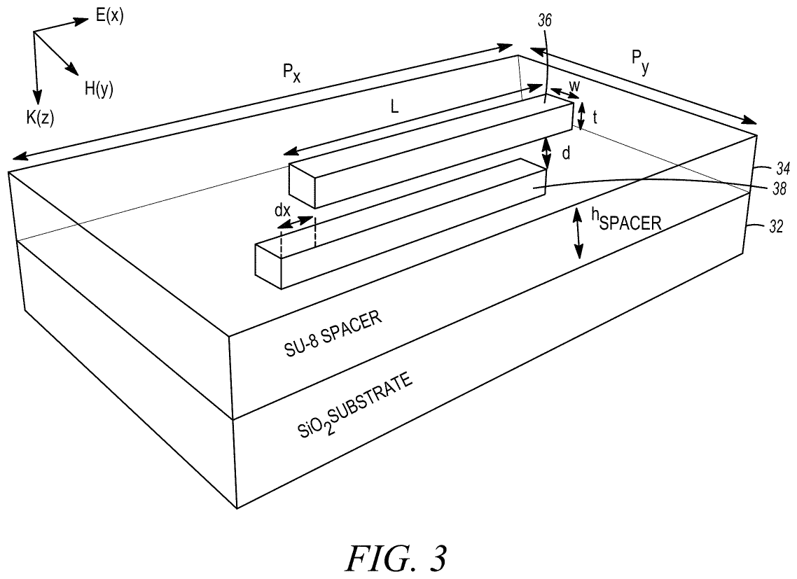

Referring next to FIG. 3, a system similar to that of FIG. 1 is illustrated, but in the case where the exceptional point is realized with only two bars 36 and 38. A substrate 32 is shown, along with the spacer layer 34. Px is the period of the unit cell in the x-axis, and Py is the period of the unit cell in the y-axis. L is the length of the nano rod, W is the width of the nano rod, t is the thickness of the nano rod, d represents the distance between two bars and dx is the shift in x. Hspacer is the spacer thickness. Implementing exceptional points with the two bar system can generally greatly simplify fabrication. By coupling two metallic bars that are in an asymmetric environment due to the presence of the substrate or that are intentionally asymmetric by using bars of different size, an exceptional point can also be attained. Modes are not of orthogonal symmetries due to the asymmetry of the system and thus can reach an exceptional points when the coupling is controlled. The two bar system has the advantage of minimizing the number of fabrication steps and greatly simplifies the implementation of the device. The two bar system can also be manufactured using self-assembly methods. The amount of loss at the exceptional point can be controlled with the number of layers and the number of layer can thus be chosen based on the loss acceptable for given applications.

The multilayered periodic plasmonic structures described above implement a plasmonic EP to reach a critical complex coupling rate resulting in the simultaneous coalescence of resonances and loss rates. The plasmonic EP enables enhanced sensing of a wide variety of substances such as analytes, which will be demonstrated below by the sensing of anti-Immunoglobulin G, the most abundant immunoglobulin isotype in human serum. In this way a new class of compact nanoscale sensors and imagers is provided based on topological polaritonic effects.

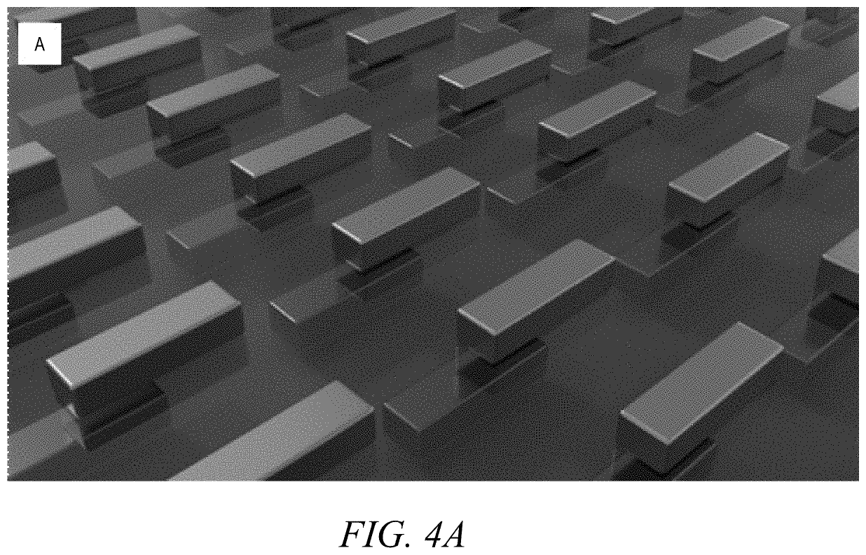

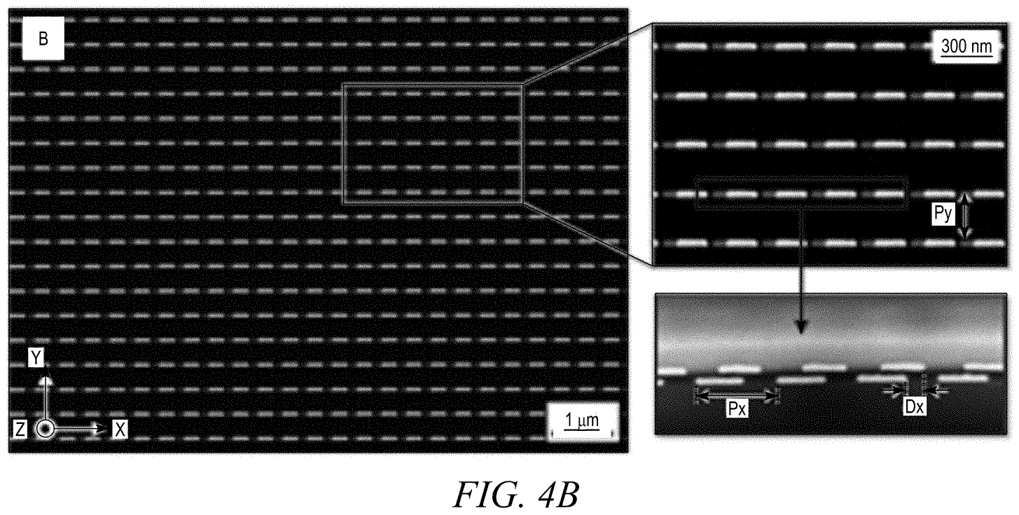

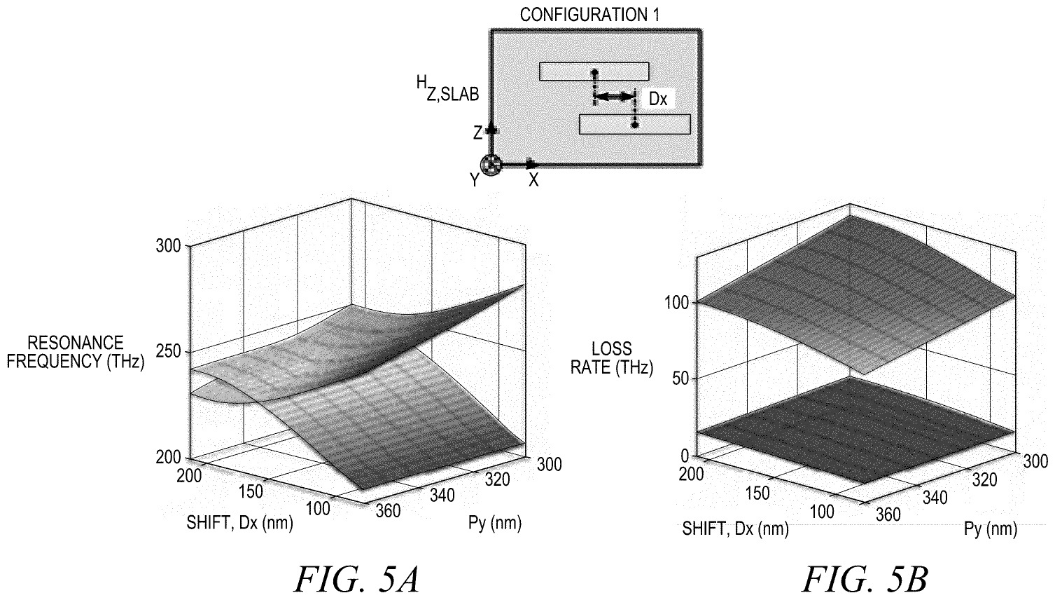

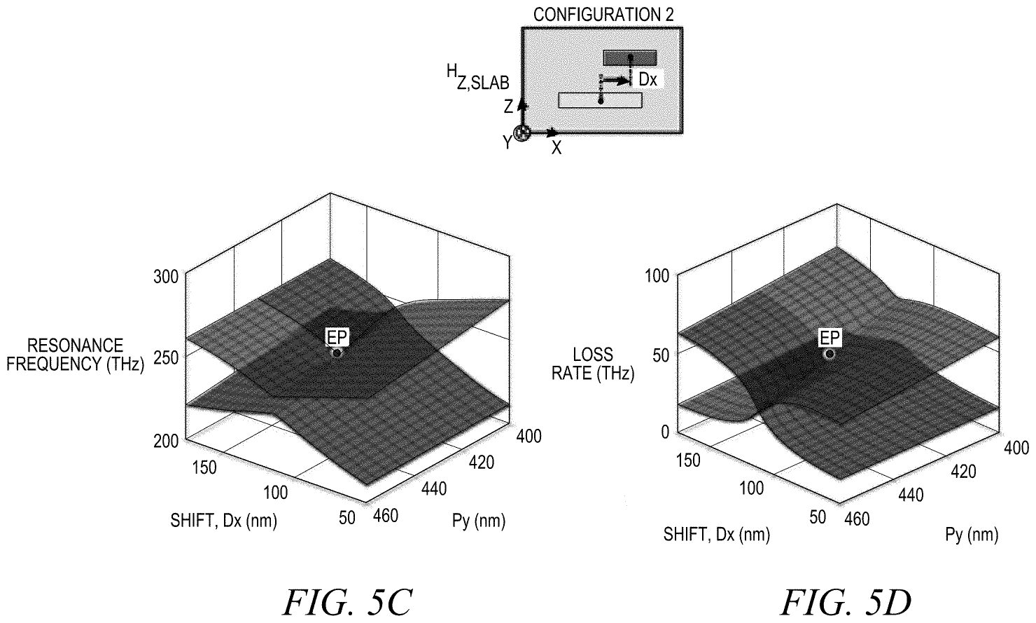

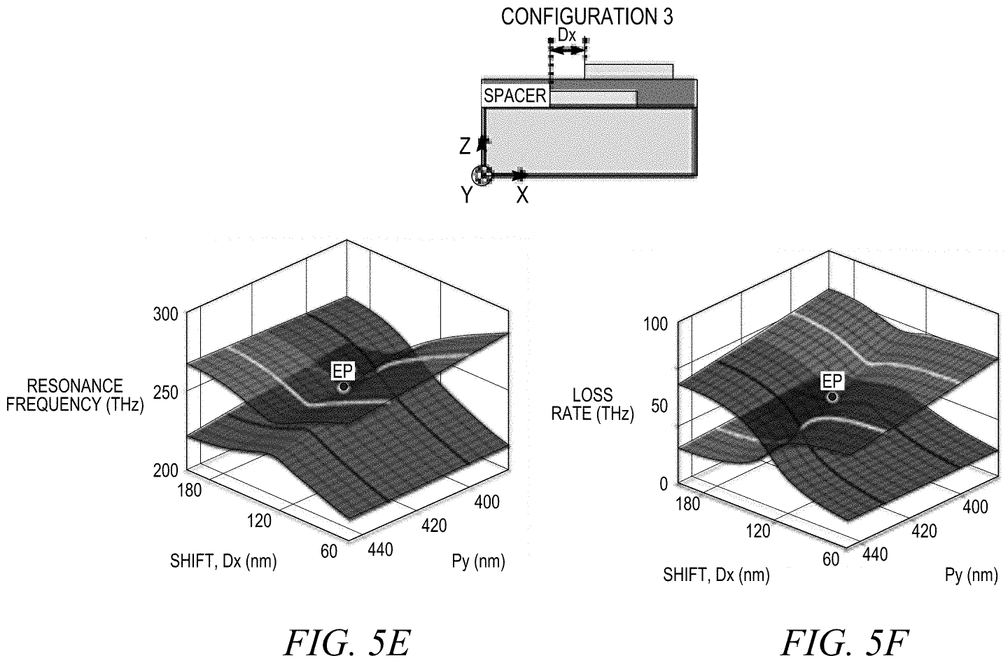

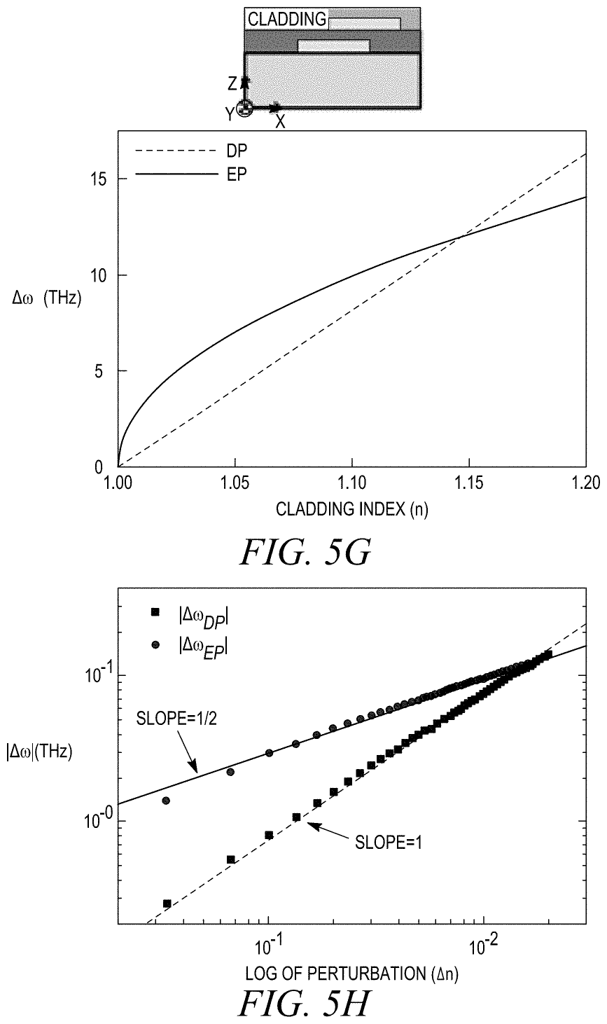

FIG. 4a is a schematic diagram of one example of a sensor that employs a bilayer plasmonic crystal similar to the type described above in FIG. 3, which is made of two optically dissimilar plasmonic resonators with detuned resonances. The periodic system is infinite in XY plane and of finite size out-of-plane and is thus a metallo-dielectric photonic crystal. The detuning is implemented by symmetry breaking either using identical resonators in distinct optical environments (FIG. 4) or using structures of distinct size in a uniform optical environment (FIG. 5). In the schematic diagram of FIG. 4a, one of the resonators is on glass and embedded in a 100 nm thick polymer spacer (SU-8) while the second resonator is on top of the polymer and is exposed to air, making its surface available for analytes binding. The gold metallic bars have a length l=250 nm, a width w=50 nm, and a thickness of t=40 nm. The structures are fabricated as described above using two steps electron beam lithography (EBL) and metal lift-off on a glass substrate (n_{sub}=1.5). The metal array patterns were defined in the bilayer e-beam resists followed by metal deposition and a lift-off process. Then, a 100 nm thick SU-8 2000.1 (MicroChem) was spun over on the first metal array as a planarized dielectric layer (n_{SU-8}=1.57). Finally, the second layer was fabricated using the same method but including a precise alignment process.

FIG. 4b (left) presents a top-view scanning electron micrograph (SEM) of the fabricated multilayer structure, with a lateral shift between bars dx=100 nm. FIG. 4b (right) shows zoom-in top (XY-plane) and side (XZ-plane) views of the plasmonic structure, clearly showing the top and bottom metallic bars and the quality of the fabrication and alignment processes. The side view image is obtained using a dual-beam focused ion beam (FIB)-SEM that simultaneously enables the local sectioning (with the FIB) and imaging (with SEM) of the samples. Px and Py are in-plane periodicities and dx is the lateral shift between the center of the bars along the direction of their electric dipolar mode (X-direction). The period along the X-direction is fixed to Px=400 nm while Py and dx are the two parameters used to tune the coupling between resonators array to reach an exceptional point. The periodic platform described herein provides stronger output signals compared to single resonator approaches and is thus more suited for a variety of applications.

FIG. 5 illustrates a plasmonic exceptional point and symmetry-dependent hybridization scheme of resonances and loss rates for various configurations. In particular, FIGS. 5a-5f present the real and imaginary parts of the eigenmodes of hybridized plasmonic arrays of optically identical resonators, configuration 1 (FIG. 5a-5b) and optically dissimilar resonators as a function of the lateral shift between the center of the dipoles (dx) and the periodicity perpendicular to the electric dipole moment (Py). The optically dissimilar structures are implemented using either resonators of dissimilar size, configuration 2 (FIG. 5c-5d) embedded in a dielectric slab (n_{slab}=1.5 and h_{z,slab}=240 nm) or using resonators of identical size in distinct optical environments, configuration 3 (FIG. 5e-5f). The hybridization of optically identical resonators leads to symmetric and antisymmetric modes, and, resonances cross along a diabolic line as a function of dx and Py (FIG. 5a). Because of the opposite symmetry of the hybridized modes, their loss rates (FIG. 5b) are very different and are always avoided. The symmetric configuration can thus not lead to exceptional points (EPs) and leads to usual Fano resonances where two modes of distinct loss rates overlap.

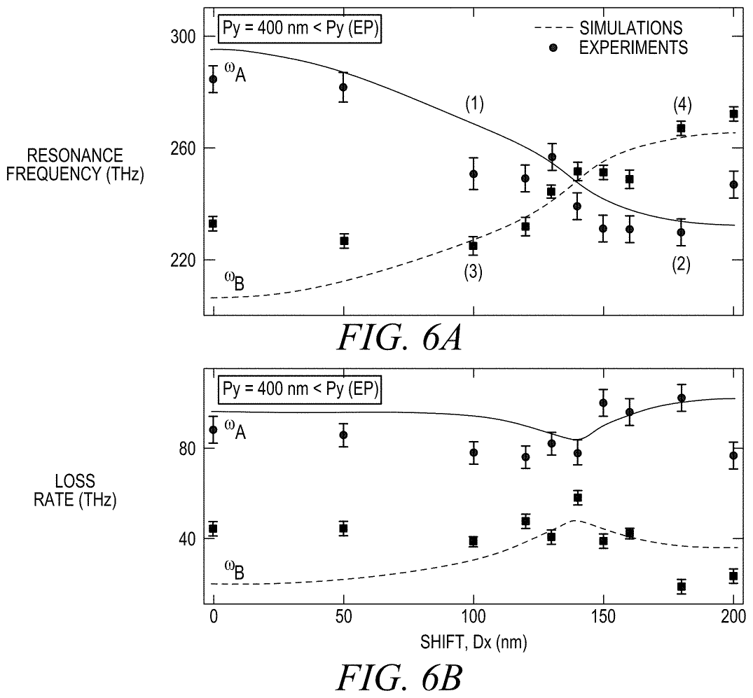

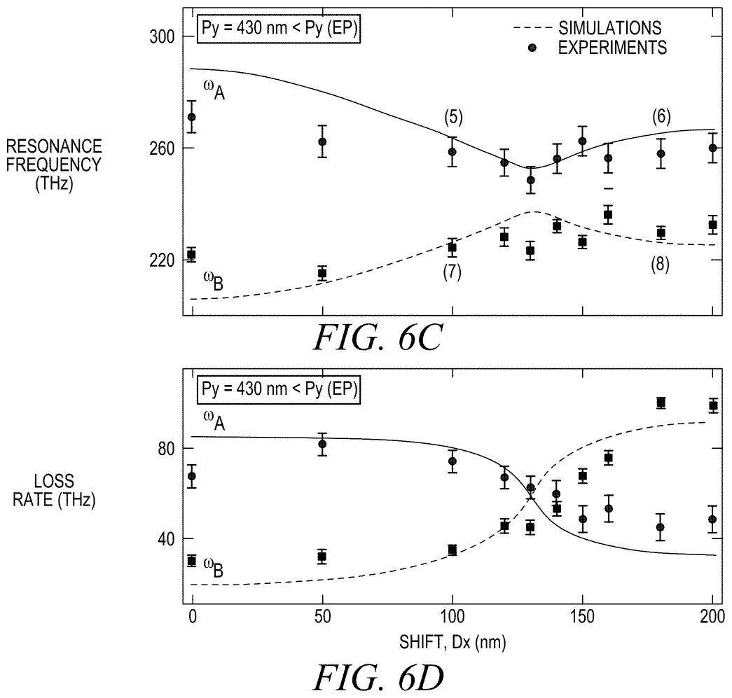

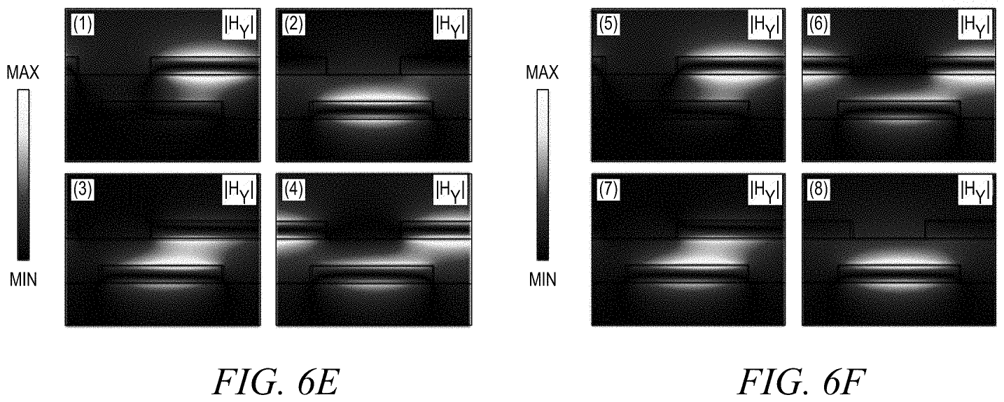

The hybridization of optically dissimilar resonators, however, leads to two hybrid modes with crossing and avoided crossing of both the resonances and loss rates (FIG. 5c-5d, 5e-5f), unambiguously demonstrating the existence of a plasmonic exceptional point where resonances and loss rates become simultaneously degenerate. By breaking the symmetry, i.e., making the bars optically different, the hybridized modes are no longer purely symmetric or anti-symmetric making interference via radiation possible. The shift of the bars mostly controls their near-field interaction while the distinct size mostly controls their far-field interference. It is worth noting that the loss rate for plasmonic EPs includes losses by radiation and absorption. The EP singularity (dot) occurs at ˜241 THz in configuration 2, and, at ˜246 THz in configuration 3. The interplay between near-field Coulomb interactions (mostly controlled by dx) and radiative coupling via interferences (enabled by symmetry breaking and mostly controlled by Py) enables the coalescence of the hybrid modes. A coupled mode model shows that, for detuned resonators, there exists a coupling rate (critical coupling) that equates the complex frequency of the two modes (see SI). The lines in FIG. 5e-5f indicate the configurations characterized experimentally in FIG. 6 to prove the existence of an EP.

To further investigate the topology of the plasmonic EP, the dispersion of plasmonic modes is analyzed around the singularity. FIG. 5g presents numerical simulations of structures (nanosensors) supporting a plasmonic EP and DP (both in configuration 3) with a cladding layer described by a varying refractive index constituting the perturbation δ. Dimensions are chosen to support either a plasmonic exceptional point (EP) [Px=400 nm, Py=415 nm, dx=134 nm] or a diabolic point (DP) [Px=400 nm, Py=350 nm dx=161 nm]. As seen in FIG. 5g the nanosensor operating around an EP exhibits resonance splitting (Δω) proportional to the square-root of the perturbation Δω_{EP}˜\sqrt{δ} whereas the DP nanosensor exhibits resonance splitting that depends linearly on the perturbation Δω_{DP}˜δ. The power laws are confirmed by log plots in FIG. 5h with slopes of 0.5 and 1.0 for the EP (Px=400 nm, Py=415 nm, dx=134 nm) and DP (Px=400 nm, Py=350 nm dx=161 nm) nanosensors respectively. The square root dependence on the perturbation constitutes an additional evidence of the successful implementation of a plasmonic EP. An analysis of the scaling of modes was performed by localizing the index perturbation to the metallic bars. Results demonstrate that the scaling laws are unchanged because the plasmonic field is essentially confined around the metal.

To experimentally demonstrate the existence of a plasmonic exceptional point, the structures of FIG. 4 are characterized as a function of the lateral shift dx for two different values of the period in the Y-direction, Py=400 nm and Py=430 nm (see the lines in FIGS. 5e-5f). FIGS. 6a-6d presents experimental (circles) and simulated (dashed curves) resonance frequencies (ω) and loss rates (γ). A very good agreement is obtained. In FIGS. 6a-6b, we observe a crossing of resonance frequencies (ω_{A} and ω_{B}) and an avoided crossing of loss rates (γ_{A} and γ_{B}) for Py=400 nm. In FIGS. 6c-6d, an avoided crossing of resonance frequencies (ω_{A} and ω_{B}) and a crossing of loss rates (γ_{A} and γ_{B}) are observed for Py=430 nm. An EP singularity thus unambiguously occurs around ˜243 THz for dx of ˜134 nm and for Py between 400 nm and 430 nm. The experimental results are obtained by measuring both the amplitude and the phase of the transmitted light. The transmittance is measured using a Fourier-transform infrared spectrometer (Bruker Vertex 70) combined with an infrared microscope (×15 Cassegrain objective, numerical aperture NA=0.4, infrared polarizer, quartz beam splitter) while the phase is measured using a custom spatially and spectrally resolved broadband interferometer.

It is worth noting that the ambiguity on whether modes are crossing or avoiding each other as one approaches the singularity can be lifted using the numerical and experimental residues around the EP. The absolute value of the out-of-plane component of the magnetic field of the modes (|Hy|) is presented in FIGS. 6e (for P_{y}=400 nm) and 6f (for P_{y}=430 nm) at indicated points on the resonance frequency dispersion plots. The out-of-plane magnetic field at points (1), (2) in FIG. 6e and points (5), (8) in FIG. 6f is mostly localized in one bar, corresponding to a dominating current flowing in that bar, and thus corresponding to an electric dipolar mode. Points (3), (4) in FIGS. 3e and (6), (7) in FIG. 6f are dominated by a magnetic field in the spacer, i.e., an antisymmetric current flow in the bars, and thus corresponding to a magnetic dipole/electric quadrupole moment. The multipolar identification of modes clearly shows that, as expected around an EP, modes are maintained as they resonances cross in FIG. 6a and switched as resonances avoid cross in FIG. 6b. The localized nature of the plasmonic resonances is better seen from the normal component of the electric field (|E_{z}|) of the modes at the same points.

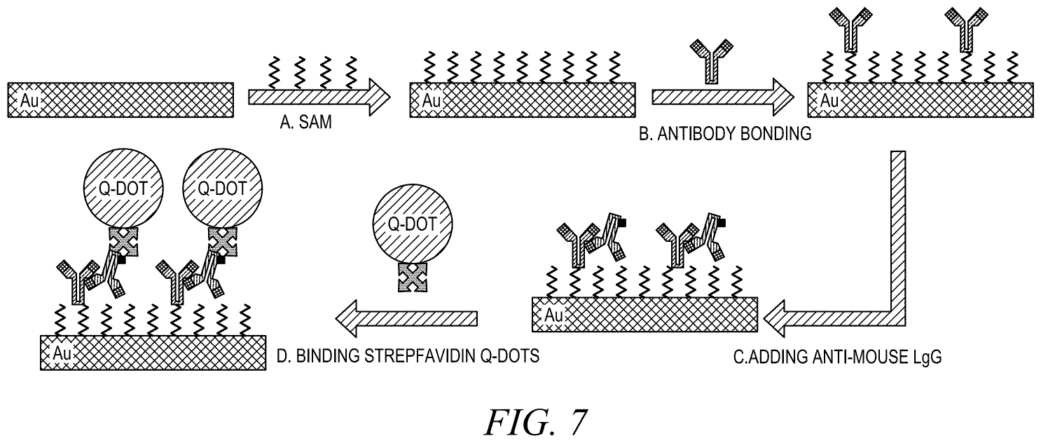

Because plasmons, the collective oscillation of free electrons coupled to photons, shrinks the wavelength of light to electronics and molecular length scales, plasmonic Eps are particularly suitable for conducting nanosensing. This will be demonstrated below by evaluating immuno-assay nanosensing using conventional Diabolic Point (DP) nanosensors and the EP nanosensors described herein. To evaluate their use in nanosensing, the top gold bars in fabricated DP and EP nanosensors of the type shown in FIG. 4 were functionalized to provide a receptor for anti-Mouse IgG. This process is illustrated in FIG. 7. First, at FIG. 7a, the gold bars were first coated with a self-assembled monolayer (linker) by submerging a clean device in an ethanolic solution of 0.1M 8-Mercaptooctanoic acid (MOA) overnight at 4° C. The device was then activated by standard EDC/NHS (Ethyl-3-(3-dimethylaminopropyl)-carbodiimide/N-hydroxysuccinimide) chemistry. Briefly, the carboxyl ends of the MOA were activated by reaction with EDC (0.4M) and NHS (0.1M) in 4-morpholino ethane sulfonic acid (MES) buffer at pH 6.5 for 35 minutes. As shown at FIG. 7b, after drying, the device was incubated with 100 μL/mL anti-CD63 antibodies for one hour at room temperature. The surfaces were subsequently blocked with 5% bovine serum albumin (BSA) in phosphate-buffered salines (PBS) for 30 minutes. Next, at FIG. 7c, after rinsing with PBS, the device was immersed in anti-Mouse IgG of a given concentration overnight at 4° C. Streptavidin Q-dots were used to verify the antibody immobilization at FIG. 7d.

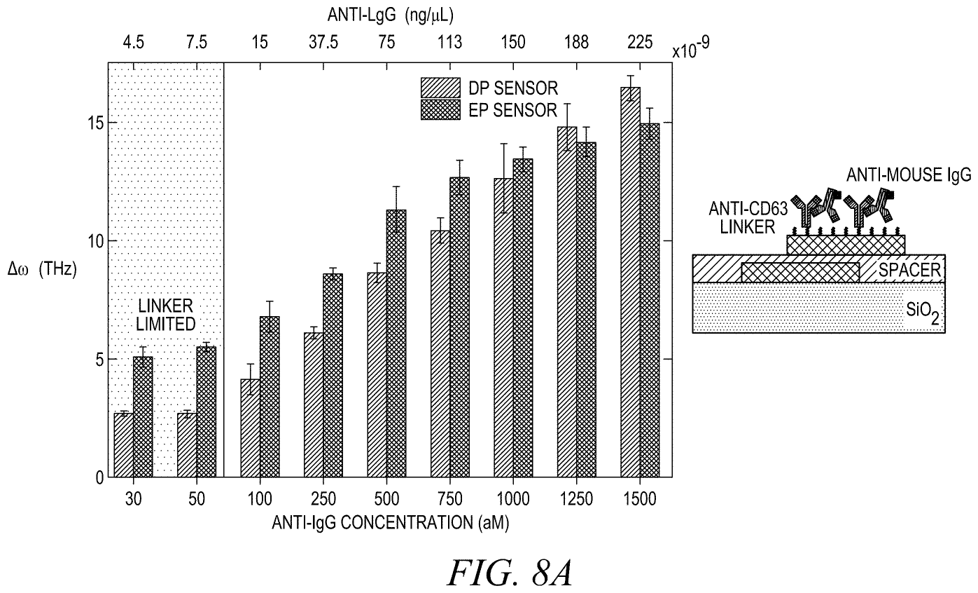

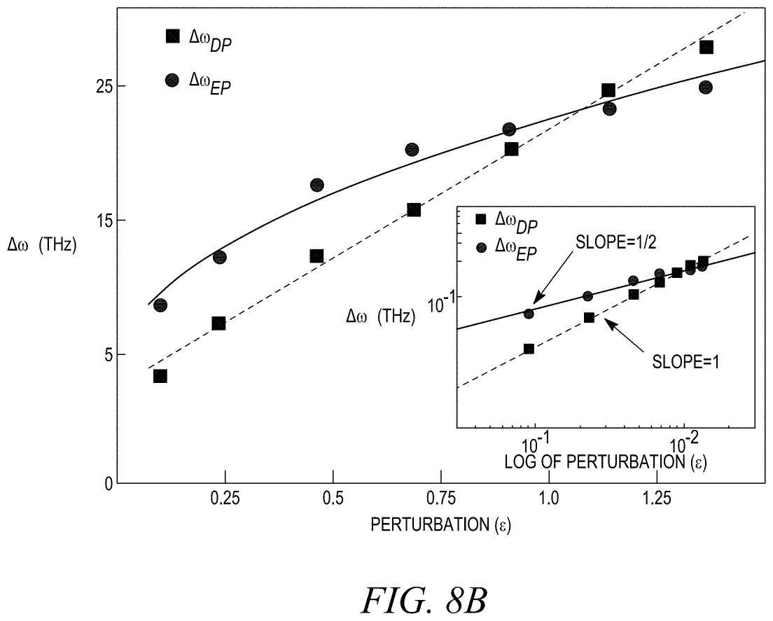

FIG. 8a are histograms presenting the measured resonance splitting for different concentrations of anti-Mouse IgG for the DP and EP sensors all in configuration 3. It should be noted that the DP samples are on the same substrate guaranteeing the same functionalization condition. Using the same sample, which is functionalized, cleaned, and functionalized again, various concentrations are compared. Starting with 1500 aM, larger splitting for the DP nanosensor is first observed (Δω_{DP}>Δω_{EP}). The concentration of IgG was then progressively decreased and larger splitting of resonances was observed for the EP nanosensor compared to the DP nanosensor for concentrations smaller than 1 fM (Δω_{EP}>Δω_{DP}). This confirms that the EP sensor becomes slightly more sensitive when the perturbation is small. The concentration was further reduced, and when the concentration is decreased from 50 aM to 30 aM, the splitting of the DP nanosensor does not change anymore. At the same concentrations, the splitting of the EP nanosensor is still larger than that of the DP sensor, but, the change in the splitting from 50 aM to 30 aM is also small. Accordingly, the EP sensor is more sensitive than the conventional DP sensor for small concentrations of analyte.

To understand this saturation, the splitting was measured for the sample solely covered with linker. The measured splittings are about 2.4 THz for the DP sensor and about 5.1 THz for the EP sensor, coinciding with the minimum splittings measured with IgG concentrations of 50 aM and 30 aM. We thus concluded that the smallest concentration of IgG that can be measured with our system has been reached. At those concentrations, sensing is thus limited by the linker that itself constitutes a perturbation while 50 aM and 30 aM or smaller concentrations of IgG in the complex linker/IgG are similar perturbations dominated by the more perturbative linker. It is worth noting that a fabrication error of ±5 nm (resolution of our fabrication) on dx for example, for the sensor exactly at the EP, already induces a mode splitting of about 6 THz, comparable to the linker induced shift (see SI). The linker can thus be neglected for concentrations between 100 aM and 1500 aM, and, we plotted, in FIG. 8b, resonance splitting as a function of IgG concentration that can be regarded as an index perturbation. Interestingly, the DP and EP mode splitting obey a linear and square-root law respectively, constituting a direct demonstration of a plasmonic EP sensor. Plasmonic sensors are usually compared using their sensitivity per refractive index unit in various regimes determined by the minimum molecular weight they can detect. The nanohole array was reported with a sensitivity of 671 nm/RIU and detected sub-10⁻⁶ g/L concentrations. The hyperbolic metamaterial was reported with 30000 nm/RIU sensitivity for a minimum detection of 2440×10⁻¹² g/L. In comparison, the EP system described herein has a sensitivity of 4821 nm/RIU and detects 15×10⁻¹² g/L and thus operates in a regime not reached by previous plasmonic array sensors.

In conclusion, plasmonic EPs can be systematically implemented by controlling the interplay between near-field and far-field couplings in hybridized systems governed by Coulomb interactions and interferences, respectively. The plasmonic EP structure, made of passive coupled arrays of symmetry-breaking plasmonic resonators with detuned resonances, exhibit the dispersion of exceptional points around the non-Hermitian singularity. The ability to drive plasmons to EPs will enable the exploration of their topological physics at small scales, such as asymmetric mode switching or Berry phase upon encircling, as well as sensors and optoelectronic devices based on topological polaritonic effects.

While the sensors herein have been described as employing EPs in plasmonic systems, more generally the sensors may employ EPs in a wide variety of different polaritonic systems in which coupled polaritonic structures are arranged to provide polaritonic resonances. Moreover, while one particular plasmonic system has been described herein in which the plasmonic stuctures are gold bars, those of ordinary skill will recognize that other materials (e.g., metals) with other shapes and configurations may be employed to provide a sensor system that is able to operate at an EP singularity.

The sensors described herein may be employed in multiple industries. For instance, they completely fit the nanotechnology requirements in applications such as electroencephalography, detection, and miniaturized devices to be used in hazardous environments. In addition, the sensors have other advantages as well, in certain implementations, e.g., may be more compact, slimmer, lossless, lighter, and potentially wearable.

Although the subject matter has been described in language specific to structural features and/or methodological acts, it is to be understood that the subject matter defined in the appended claims is not necessarily limited to the specific features or acts described above.

1. A method for detecting an analyte, comprising:
providing a sensor that includes a plurality of coupled polaritonic structures having polaritonic resonances;
functionalizing a surface of at least one of the polaritonic structure in the sensor by providing a receptor for binding the analyte to the surface;
operating the sensor at an exceptional point (EP); and
identifying a presence of the analyte on the surface when a degeneracy of resonant frequencies and linewidths is lifted and a splitting of the resonant frequencies and linewidths occurs.

2. The method of claim 1, wherein the plurality of coupled polaritonic structures is arranged as a multilayer structure.

3. The method of claim 2, wherein the plurality of coupled polaritonic structures is arranged as a bilayer structuere.

4. The method of claim 2, wherein the plurality of coupled polaritonic structures is arranged as a plasmonic structure.

5. The method of claim 4, wherein the plasmonic structures are formed from a metallic material.

6. The method of claim 5, wherein the metallic material includes gold.

7. The method of claim 1, wherein each of the polaritonic structures are nanoscale structures.

8. The method of claim 1, wherein the operating includes controlling symmetry compatible modes.

9. The method of claim 4, wherein the operating includes controlling symmetry compatible modes via near field and/or far field interactions.

10. The method of claim 3, wherein the modes are hybridized modes.

11. The method of claim 2, further comprising a dielectric spacer disposed between layers of the multilayer structure.

12. The method of claim 1, wherein the operating includes operating at the EP based on the hybridization of detuned resonators in the coupled polaritonic structures.
